(19) United States
(12) Patent Application Publication
(10) Pub. No.: US 2005/0100201 A1
(43) Pub. Date: May 12, 2005

(54) Device for monitoring an operating parameter of a medical device

(76) Inventors: Robert Mayer, Lauf/Schonberg (DE); Norbert Karl Strobel, Palo Alto, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/972,313

(22) Filed: Oct. 22, 2004

(30) Foreign Application Priority Data
Oct. 24, 2003 (DE) 10349661.0

(51) Int. Cl. G06F 19/00; G01N 33/48; G01N 33/50; G06K 9/00

(52) U.S. Cl. 382/128; 702/19

(57) ABSTRACT

The present invention relates to a device and a method for monitoring parameter selection during the operation of a technical device, in particular an imaging diagnostic device. The device comprises an input interface (1) for a parameter selected by an operator of the technical device, a comparator (2) which compares the selected parameters with the standard parameters, and an output device (3) which, in the event of a deviation of the selected parameters by a predefinable minimum degree from the closest standard parameters, outputs information regarding the deviation for display on a display unit (4) and/or outputs the closest standard parameters for adjustment of the technical device. Improved results are achieved with the present device and the associated method during operation of the technical device.

### CROSS REFERENCE TO RELATED APPLICATIONS

This application claims priority to the German application No. 10349661.0, filed Oct. 24, 2003 and which is incorporated by reference herein in its entirety.

### FIELD OF INVENTION

The present invention relates to a device and a method for monitoring parameter selection during the operation of a technical device, in particular an imaging diagnostic device.

### BACKGROUND OF INVENTION

The device and the method should thereby primarily be assigned to the field of medical imaging devices. Nevertheless they can also be used without more ado for operator support with other technical devices, wherein the operator has a large degree of freedom when selecting the operating parameters, even though the quality of the operating result, in other words, the output of the technical device, essentially depends on the appropriate selection of said parameters. This is illustrated below with reference to application in radiological imaging examinations.

In the case of radiological examinations, with an X-ray CT device for example, imaging should be controlled such that an optimal image quality is achieved with the minimum dosage load for the patient. There are a large number of operating parameters for this type of device, which can be adjusted by the operator and which are established in the form of a measuring protocol. Thus, for example X-ray power, rotation time, layer thickness, feed per rotation, different kernels and further mechanical parameters or parameters required for image reconstruction can be selected. Other parameter combinations can be selected respectively for different examination regions of the body and different purposes of the corresponding image recording in order to achieve an optimum image result. Knowledge of the underlying relationships and sufficient experience with devices of this type are required for this.

### SUMMARY OF INVENTION

With the introduction of new X-ray imaging products, priority is given to training medical radiology assistants (MTRAs) in order to operate the devices. The relevant doctors, e.g. radiologists or cardiologists then familiarize themselves with the devices. In practice the device settings are however frequently predefined by the respective doctors, without allowing the knowledge of the MTRAs to have any influence. Because they are less well informed about the new products, this regularly results in sub-optimum device settings and thus unsatisfactory image quality. It has hitherto not been possible for the manufacturer of the corresponding device to recognize and correct such inappropriate behavior.

An object of the present invention is thus to specify a device and a method for monitoring parameter selection during the operation of a technical device, the deployment of which results on average in an improved result during the operation of the device. The device and the method should in particular make it possible to supply optimum image results with technical imaging devices for the application in question.

This object is achieved by the claims. Advantageous embodiments of the device and the method are the object of the dependant claims or will emerge from the subsequent description and the exemplary embodiments.

The present device for monitoring parameter selection during the operation of a technical device comprises an input interface for parameters selected by the operator of the technical device, a comparator, which compares the selected parameters with standard parameters, and an output device which, in the event of a deviation of the selected parameters by a predefinable minimum degree from the closest standard parameter, outputs information regarding the deviation for presentation on a display and/or outputs the standard parameters closest to the selected parameters for adjustment of the technical device. In the first instance, the operator is able to adjust the selected parameters to the standard parameters and/or to adopt the standard parameters, whereas in the second instance the parameters selected by the operator are automatically replaced by the closest standard parameters.

The standard parameters can thus be retrieved by the comparator for example from a database, which is a component of the device. In one embodiment of the device, the comparator comprises a communication interface to establish a network connection with a corresponding database, from which the standard parameters are retrieved. This network connection can for example also be established via the internet, whereby the database can be kept available for example on a server belonging to the manufacturer of the technical device.

As a result of the generally diverse possibilities of parameter s election for different applications, different standard parameter sets are available, which are tailored to these different applications. When executing a comparison the comparator only uses the closest parameter set(s), since these most probably correspond to the mode of operation desired by the operator.

With the present device and the method underlying said device, the operator is able to optimize the result with the aid of the experience of the manufacturer of the technical device and other experts who created the standard parameters. It is precisely in the area of technical imaging devices, for example in medical imaging diagnostics, that image results with superior image quality are reliably achieved by means of the present device.

The present device can thereby be implemented for example directly in the technical device or even at a workstation connected to the technical device, in the case of a medical imaging device for example the findings station. It is precisely with imaging devices for medical diagnostics that the operator can be provided with recommended values, in other words standard parameters and indications, by means of the present device and the method associated therewith, prior to executing the image recording in order to improve the settings by comparing the selected imaging parameters (e.g. scan protocol parameters). With devices of this type, it is also possible to provide suggestions for improving the image results with parameter selection for the reconstruction of images from the measured values, even after the execution of the actual image recording. The operator is shown the corresponding information on a monitor, on which the parameters are selected and/or the findings are given.

In a very advantageous embodiment of the present invention and the method associated therewith, the operator is shown the deviations from an optimum mode of operation resulting from their parameter selection by means of images. This can be achieved with imaging devices, in that the user is shown one or a plurality of sample image results on the display device, which are received using one or a plurality of standard parameter sets closest to the selected parameters. The sample images can hereby be stored in a database, together with the associated standard parameter sets. At the same time the image result is simulated using the sample displayed, showing what would result with the in some instances sub-optimum parameters selected by the user. This second image result is compared with the first sample image result(s), so that the user can directly identify the quality differences in the image result. The device is thereby configured such that the operator can adopt the associated parameters to adjust the technical device simply by selecting such an image result.

The last-mentioned embodiment can not only be achieved with technical imaging devices, but also with other technical devices, such as those used for material processing. In this case, an image of a workpiece sample processed using optimum parameters can be compared with a simulated representation of workpiece samples obtained using the selected parameters in the display.

The different standard parameter sets are preferably available as feature vectors in a multidimensional parameter space, subsequently referred to as the feature space. It can be advantageous here if different feature spaces are defined for different applications of technical devices, from which the operator can then make a selection by predefining the corresponding application. The standard parameter vectors present in the respective feature space are as a rule determined by the manufacturer of the technical device. The procedure linked to the formation of the feature spaces and the standard parameter vectors and the subsequent comparison reverts back to the basic principles of object classification.

The parameters selected by the operator of the technical device for the specific application are thereby also represented as feature vectors and are input in the corresponding feature space as a function of the type of the respective application and the device used. Distance values can then be determined in respect of available standard parameter vectors whereby it can be advantageous to weight the individual parameters differently during the distance calculation. The distance calculation is automatically carried out by the present comparator. Information, for example references to associated sample protocols and displays of sample images, is then provided as a function of the distance value(s) determined.

As well as the operator prompt displayed, the feature vectors resulting from the operator settings also make it possible to analyze the device settings adjusted by the operator. An analysis of this kind can be carried out both locally with reference to the respective device and globally by evaluating a plurality of operator settings which were carried out at different devices of the same device type at different locations. Personal operator preferences can therefore result at specific locations or devices in the parameters selected by the operator for different application and systems deviating from the standard parameters. In the event that the deviations are unusually large, a specific intervention can take place locally and counter controls can for example be set in place by corresponding training. With global notification via a plurality of devices, multidimensional distribution densities can be obtained in this way in the feature spaces. These distribution densities should have clusters, the centers of which should correspond to the standard parameter vectors. Should this not be the case, in other words if the cluster center deviates from the standard parameter vectors, possible systematic errors can be investigated in the definition of the standard parameters.

In any case an evaluation of the actual user settings which are stored as feature vectors makes it possible with the added benefit of classification technology to verify the use of the technical devices in practice. In the case of diagnostic imaging devices, this also allows ongoing quality control, whereby the relationship between the imaging device used, the imaging parameters selected by the operator, the radiological findings results, the associated overall diagnosis and final treatment results among other statistically aspects could play a role.

In a development of the device, a training module can also be integrated thereby enabling computer-based training. This training module enables the operator to change certain parameters and to display the effect of this change on the operating result of the technical device on the display unit. This training module can for example be used in the case of CT applications, to indicate to the operator how the scan parameters “ImaIncrement”, “EffectiveSliceWidth” and “Kernel” affect an MPR representation. The operator can thereby be offered a sliding control on the screen for example for relevant imaging parameters. If the sliding control is moved, the resulting sample image is displayed in real-time. This module enables the operator to define and store their own reference imaging parameters by means of simulation, so that they can retrieve said parameters whilst the device is in operation.

### BRIEF DESCRIPTION OF THE DRAWINGS

The present device and method associated therewith are described below with reference to an exemplary embodiment in conjunction with the drawings, in which

FIG. 1 shows an illustration of a three-dimensional feature space with parameter vectors defined therein;

FIG. 2 shows a schematic representation of a possible embodiment of the present device as a block diagram, and

FIG. 3 shows an example of the representation of information regarding the deviation on a display unit.

### DETAILED DESCRIPTION OF INVENTION

The present exemplary embodiment relates to the use of the present device and the method associated therewith in medical imaging using an X-ray CT device. The present device, as shown in FIG. 2 with reference to an exemplary embodiment, is implemented in this case at the findings station, a workstation linked to the CT device. The operator uses this findings station to input the imaging parameters required for the imaging to be recorded or selects these at the findings station.

A database 5 is also implemented at this findings station, wherein the standard imaging parameter sets (sample protocols) of relevance to the connected device, are defined using standard imaging parameter vectors in different feature spaces. The different feature spaces are thereby provided for different diagnostic examination applications. For example, the following information can play a role in the definition of the feature spaces:
modality (CT, MR, US, AX etc.) and specific device types (e.g. Somatom Sensation, Somatom Emotion, Somatom Smile);
Patient characteristics such as gender, height or weight;
Area of body being examined, for example, head;
Part of the body in question, for example, brain;
Type of examination arranged, for example CT angiography
Selection of imaging parameter protocols, for example organ programs

The standard imaging parameter vectors form the key points of imaging parameter classes and represent these in the associated feature spaces. All the standard imaging parameters together form what is referred to as the imaging knowledge base, in the present case the database 5. A sample image data set is preferably stored for each imaging parameter class generated in such a way. This sample image data set shows the image quality, which can be achieved with the associated standard imaging parameter settings (related to a sample case). The comparator 2 of the present device has access to the database 5 either directly or—in the case of an external database 5—via a corresponding communication interface 6 for the establishment of the network connection (see FIG. 2).

By implementing the database 5 in the present device, updates of the standard imaging parameters can be introduced either via a corresponding data carrier or via a network. An example of this is the customer care solution “Somatom Life”, as known for example from the publication UPTIMES, a supplement to icare, Vol. 1/2003, Customer Service from Siemens Medical Solutions, page 8.

When using the present device, the operator at the findings station first selects the scan and reconstruction parameters which they deem suitable for the intended recording. These parameters are supplied via the input interface 1 of the device to the comparator 2, wherein the input parameters are represented as feature vectors, also referred to below as customer imaging parameter vectors, and are input in the assigned feature space as a function of the type of examination and device used in each instance.

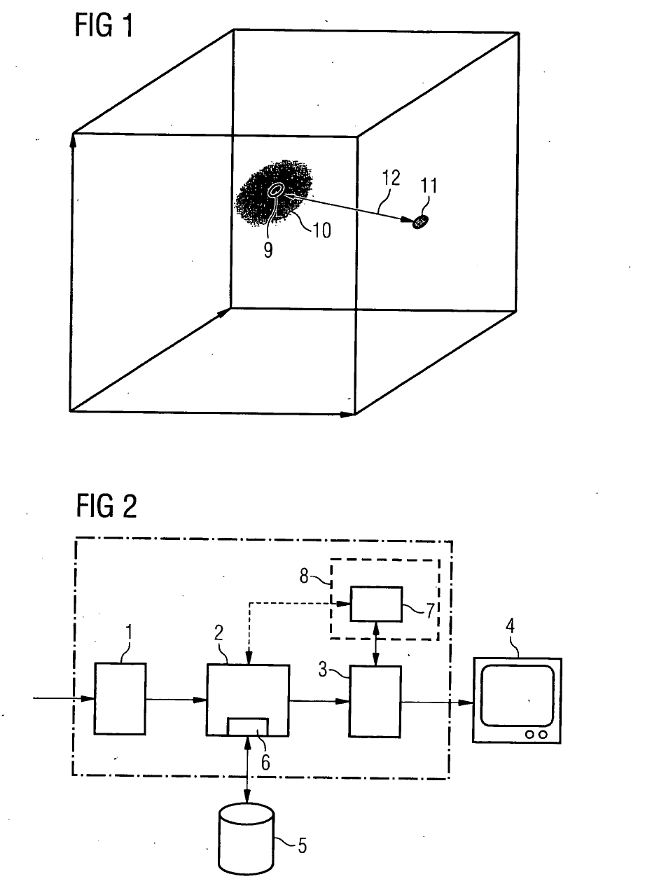

For the purposes of illustration with reference to a three-dimensional example FIG. 1 shows the spread 10 of customer imaging parameter vectors around a standard imaging parameter vector 9 (\vec{d}₀). Furthermore in this diagram a customer imaging parameter vector 11 (\vec{d}) selected by the operator is input, which is compared by the comparator 2 with the standard imaging parameter vector 9.
For this purpose the weighted distance 12 between the standard imaging parameter vector 9 and the selected customer imaging parameter vector 11 is calculated. This distance d between the two vectors \vec{d}₀ and \vec{d} is shown in FIG. 1, by means of the arrow. The distance d is thereby obtained for example using the following formula:

$$d = \text{(} {d}^{->} - {{d}^{->}}_{0} \text{)} ∘ ( \begin{matrix}{w}_{1} & 0 & 0 & 0 & 0 \\ 0 & {w}_{2} & 0 & 0 & 0 \\ 0 & 0 & ⋰ & 0 & 0 \\ 0 & 0 & 0 & {w}_{n - 1} & 0 \\ 0 & 0 & 0 & 0 & {w}_{n}\end{matrix} ) ∘ \text{(} {d}^{->} - {{d}^{->}}_{0} \text{)}$$

The individual values w₁ to wₙ of the matrix specify the different weighting factors, whereby n corresponds to the dimension of the feature space.

As a function of the determination of the distance value d by the comparator 2, indications can be given via the output unit 3 on the monitor 4 of associated sample protocols and their sample images can thus be displayed. Indications of this type can be given at the findings station both prior to and after a CT scan. In the first instance an MTRA is primarily addressed, while in the second instance the radiologist receives notification. The indication of divergent or non-optimum parameters can be displayed in a form, which corresponds to a radiological context-sensitive help device, which uses clinical cases to show how a CT scanner should be set or should have been set optimally. One example of a user prompt of this type, which can also be used with the present device, is the “Phoenix” application, which is for example known from the publication icare from Siemens Medical Solutions, Vol. 1/2003, page 40. In this way, the setting parameters, with which the displayed sample images were generated, can be adopted using a simple drag and drop process to set the specific device. For example two image results are compared on the display, the first of which displays the result, which would be achieved with the settings selected by the user. In contrast the second result conveys the image which would be achieved if the optimized settings were selected. This image could then be downloaded according to the “Phoenix” application and subsequently the scanner settings could then be automatically adjusted correspondingly.

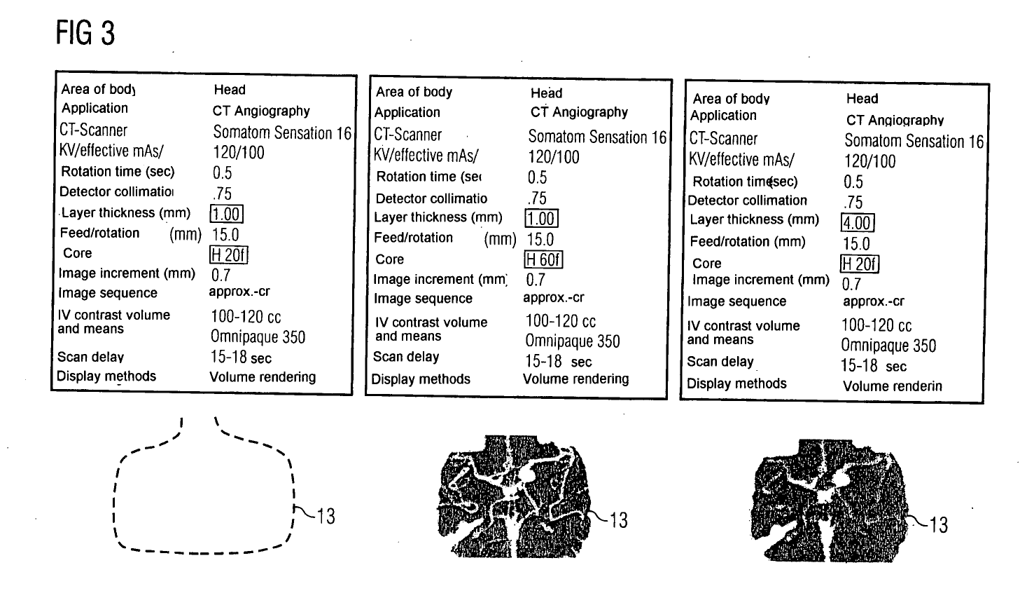

FIG. 3 shows an example of the representation of information of this type on a display unit. In the example shown the imaging parameters selected by the operator and identified on the left hand side are compared with two alternative standard parameter settings, the imaging parameter vectors of which lie at feature distance 20 and 40 from the feature vectors selected by the operator. In the example shown here the CT imaging parameters “layer thickness” and “core” do not correspond in an optimum manner. The closest standard imaging parameter (Distance 20) corresponds to a setting, as selected for reconstruction with good local resolution. The second standard parameter set (Distance 40) is for well-defined differentiation of soft tissue contrasts. The other imaging parameters s elected by the operator indicate that a good local resolution is required for this examination, which is also reflected in the smaller distance measurements in respect of the corresponding standard imaging parameter vectors. To show the image quality which can be achieved with the standard imaging parameter vectors, this is shown with reference to a sample case below the respective parameter settings as exemplary image 13. In addition, the image result anticipated on the basis of the feature vector selected by the user is simulated in the simulator 7 of the present device and is also shown correspondingly below this parameter. This is shown in FIG. 3 with a broken line. The operator is then able to select the image result most suited to their purpose, for example to record the respective image simply using drag and drop in their own workspace, thereby automatically adopting the corresponding imaging parameters for the device.

FIG. 2 also shows a training module 8, containing the simulator 7, in order to show the operator a training option for understanding the effects of specific image parameters on the image result. This was already explained in the above description.

If, after selecting such standard imaging vectors and sample protocols, the operator obtains imaging results which deviate significantly from the sample images, this indicates that the system is technically defective and must undergo a service. If selected imaging parameters differ significantly with respect to their post-processing settings, for example the CT reconstruction settings such as the filter core, from sample protocols, interventions can be made even after image acquisition and alternative post processing steps and/or settings can be indicated. The present device also offers the possibility with a suitable classification method of automatically assigning the user settings to a specific scan parameter class, which then automatically executes or corrects the imaging parameter selection in part or as a whole.

1-23. (canceled)

24. A device for monitoring an operating parameter of a medical device, comprising:
an input interface for acquiring a first value of the operating parameter selected by a user of the medical device;
a comparator for comparing the first value to a default setting of the operating parameter; and
an output device for outputting information related to a deviation of the first value from the default setting.

25. The device according to claim 24, wherein the information related to the deviation includes a second value of the deviation.

26. The device according to claim 24, wherein the information related to the deviation includes the default setting.

27. The device according to claim 24, wherein the user adjusts the medical device based on the information related to the deviation.

28. The device according to claim 24, wherein the medical device is a medical diagnostic imaging device.

29. The device according to claim 24, wherein the comparator is connected to a database containing a plurality of default settings related to a plurality of operating parameters for retrieving the default setting.

30. The device according to claim 24, wherein the comparator has a communication interface for establishing a network connection to a database for retrieving the default setting.

31. The device according to claim 24, further comprising a simulator connected to the output unit for generating and outputting a first graphic representation related to an assumed continued operation of the medical device based on the first value, wherein the information related to the deviation includes a second graphic representation related to an assumed continued operation of the medical device based on the default setting.

32. The device according to claim 31, wherein the default setting includes a tolerance band.

33. The device according to claim 24, wherein the output unit is adapted to accept the default setting upon a user request and output the default setting for adjusting the medical device.

34. The device according to claim 24, wherein the default setting is a parameter vector in a multidimensional parameter space.

35. The device according to claim 34, wherein the default setting includes a plurality of multidimensional parameter spaces corresponding to a plurality of different user-selectable applications of the medical device.

36. The device according to claim 34, wherein the comparator calculates a distance between a vector representing the operating parameter in the parameter space and the default setting, wherein the calculated distance corresponds to the deviation.

37. The device according to claim 36, wherein a minimum of the calculated distance corresponds to a minimum of the deviation.

38. The device according to claim 36, wherein the comparator calculates the distance using a plurality of different weight parameters.

39. The device according claims 34, wherein a parameter vector corresponding to the first value is automatically stored by the comparator for a subsequent analysis.

40. The device according to claim 24, further comprising a training module having a simulator for training the operator, wherein
the training module is adapted to:
adjust a parameter setting of the medical device in a simulation without influencing any operating parameters of the medical device; and
generate a graphic representation of a simulation result based on the adjusted parameter setting using the simulator, and
the graphic representation of the simulation result is output on the output device.

41. The device according to claim 40, wherein the training module is further adapted to store the parameter setting of the medical device adjusted in the simulation for subsequently adjusting the medical device.

42. A method of monitoring an operating parameter of a medical device, comprising:
providing a monitoring system, the monitoring system comprising:
an input interface acquiring a first value of the operating parameter selected by a user of the medical device; and
a comparator for comparing the first value to a default setting of the operating parameter;
providing an output screen; and
outputting information related to a deviation of the first value from the default setting on the output screen.

43. The method according to claim 42, wherein the medical device is a medical CT or MR system.

44. A method of monitoring a user-selectable operating parameter during operation of a medical device, comprising:
comparing the user-selected parameter with a default setting of the operating parameter; and
calculating a deviation of the operating parameter from the default setting.

45. The method according to claim 44, further comprising:
displaying information related to the calculated deviation on a screen, if the deviation exceeds a maximal deviation.

46. The method according to claim 44, further comprising:
adjusting the operating parameter according to the default setting, if the deviation exceeds a maximal deviation.

47. The method according to claim 44, wherein the medical device is a medical diagnostic imaging device.

48. The method according to claim 44, wherein the default setting is retrieved from a database.

49. The method according to claim 45, wherein the information related to the calculated deviation includes the default setting.

50. The method according to claim 45, further comprising outputting a first graphic representation related to an assumed continued operation of the medical device based on the operating parameter using a simulator, wherein the information related to the deviation includes a second graphic representation related to an assumed continued operation of the medical device based on the default setting.

51. The method according to claims 44, wherein the default setting is a parameter vector in a multidimensional parameter space.

52. The method according to claim 51, wherein the default setting includes a plurality of multidimensional parameter spaces corresponding to a plurality of different user-selectable applications of the medical device.

53. The method according to claim 51, wherein calculating the evaluation includes calculating a distance between a vector representing the operating parameter in the parameter space and the default setting.

54. The device according to claim 53, wherein a minimum of the calculated distance corresponds to a minimum of the deviation.

55. The method according to claim 53, wherein the distance is calculated using a plurality of different weight parameters.

56. The method according to claim 51, wherein a parameter vector corresponding to a value of the operating parameter is automatically stored for a subsequent analysis.
